(19) United States
(12) Patent Application Publication
(10) Pub. No.: US 2005/0065348 A1
(43) Pub. Date: Mar. 24, 2005

(54) Pyridin-2-yl-methylamine derivatives for treating opioid dependence

(76) Inventors: Francis Colpaert, Puylaurens (FR); Liesbeth Bruins Slot, Castres (FR); Wouter Koek, San Antonio, TX (US); Jean-Pierre Tarayre, Castres (FR); Bernard Vacher, Castres (FR)

Correspondence Address: BURNS DOANE SWECKER & MATHIS L L P, POST OFFICE BOX 1404, ALEXANDRIA, VA, 22313-1404, US

(21) Appl. No.: 10/483,567

(22) PCT Filed: Jul. 11, 2002
(86) PCT No.: PCT/FR02/02449

(30) Foreign Application Priority Data
Jul. 13, 2001 (FR) 0109350

(51) Int. Cl. C07D 211/82

(52) U.S. Cl. 546/315

(57) ABSTRACT

The invention concerns compounds of general formula (I) for treating opioid drug dependence.

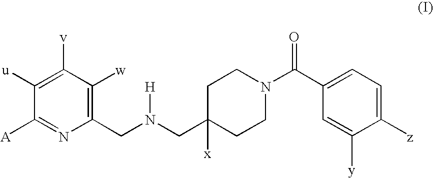

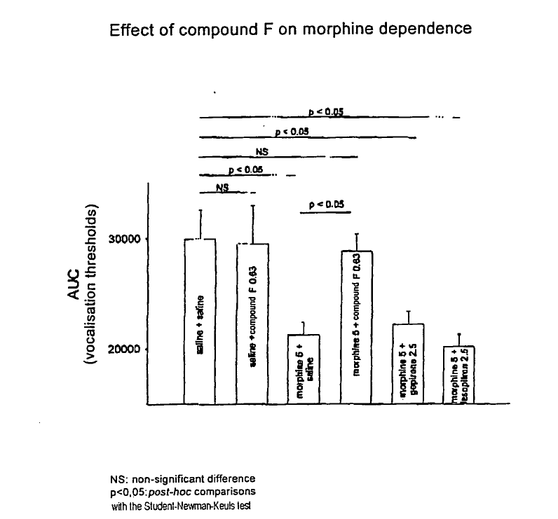

The aim of the present invention is a new utilisation of certain compounds of pyridin-2-yl-methylamine for the treatment of drug dependence.

Drug dependence on a psycho-active substance is a pathological phenomenon, classified among mental troubles in the international classification of the World Health Organisation. The list of substances producing a drug dependence or dependence syndrome is long. However, the field of application of the present invention is limited to treatment of opioid drug dependence. By opioids, the inventors mean natural, semi-synthetic or synthetic compounds as a whole, having a morphinic activity.

The principle for treating drug dependence is based on enabling the dependent subject to become, and then to remain, abstinent from the substance on which he or she is dependent. In the particular case of opioid dependence, the pharmacological treatments in use are so-called substitutive treatments. Their principle consists in replacing the substance or substances responsible for the dependence by an opioid product with lower specific effects and with a longer active period than those of the substance(s) responsible for the dependence. The medicaments available in the substitution indication are, according to the country, methadone chlorhydrate, levo-alpha-acetylmethadol (LAAM) and buprenorphine. However, the success rate of these treatments is limited. In addition, since the products used in substitution treatments belong themselves to the pharmacological opioid class, they also pose a direct or indirect risk for public health. Thus, substitution products can induce a dependence syndrome and/or increase the risk of abuse and dependence on other substances such as, for example, cocaine. Conjointly with substitution treatments, certain agonists of O₂ receptors, such as clonidine and lofexidine are sometimes also used in support and prevention treatment for relapses after opioid withdrawal therapy.

Present treatments for opioid drug dependence are therefore not entirely satisfactory. As a result, the discovery of new treatments involving molecules with a mechanism action different from that of existing products is highly desirable.

The mechanisms underlying the dependence syndrome are complex and, as a result, difficult to determine. In fact, different classes of receptors and neurotransmitters are involved. Nonetheless it seems to be established that dopamine plays a central role. It is also evident that noradrenaline and serotonine, which among other things act as modulators and/or regulators of dopaminergic neuron activity in the cerebral areas involved in dependence phenomena, constitute privileged targets for therapeutic interventions.

In fact, the existence of a link between certain sub-types of the serotonine bond site, including the 5-HT_{1A} sub-type among others, and morphine dependence, has been discussed in scientific reports since the end of the '80s. Consecutively, the potential field of application of compounds with an affinity for the 5-HT_{1A} receptor has, more or less systematically, been extended to treating withdrawal syndrome and/or abuse and/or drug dependence on psycho-active substances, including ethanol and nicotine.

Thus, in the European patent application EP 356997, the firm of Bristol-Myers Co. Claims the utilisation of azapirones such as, for example, buspirone in treating abuse of psycho-active substances.

In international applications WO 0035892, WO 0035874, and WO 0035878, the firm of American Home Products Corporation describes derivatives of piperazine-ethylamides, aryl-piperidines and 1,4-piperazines respectively, as agonists and antagonists of the 5-HT_{1A} receptor, useful in treating drug habituation. In the international application WO 9938864, the same company describes derivatives of oxazoles as agonists of the receptor 5-HT_{1A} useful in treating withdrawal syndrome and habituation to drugs. In applications WO 9808817 and WO 9717343, derivatives of 4-aminoethoxy-indoles and benzodioxane-methylamines, respectively, are claimed as ligands of the 5-HT_{1A} receptor useful in treating abuse and dependence. In the international application WO 0035875, derivatives of aryl-piperidines are claimed as antagonists of the 5-HT_{1A} receptor useful for treating drug habituation.

In the international applications WO 9723485, WO 9702269, WO 9703071, the Knoll company claims derivatives of heteroaryl-carboxamides, of thiazoles, and of heterocyclyl-carboxamides, respectively, as non-selective ligands of the 5-HT_{1A} receptor useful in treating abuse of and habituation to psycho-active substances.

In international application WO 9730050 the Pharmacia & Upjohn S.P.A. company reports derivatives of heterocyclyl-ergolines as selective ligands of the 5-HT_{1A} receptor useful in treating drug withdrawal and habituation; equally, in application WO 9741858 derivatives of piperazines are disclosed as non-selective ligands of the 5-HT_{1A} receptor useful in treating abuse of psycho-active substances.

In applications EP 982030 and EP 928792 the company of Pfizer Products Inc. discloses derivatives of 2,7-substituted-octahydro-pyrrolo-1,2-pyrazines and Bicyclo (3.1.0.) hexanes, respectively, as ligands of the 5-HT_{1A} receptor useful in the treatment of dependence.

In the international application WO 9842344 the company of R.P. Scherer Limited claims the utilisation of a pharmaceutical composition comprising a 5-HT_{1A} agonist such as, for example, buspirone, in the treatment of abuse and habituation to certain substances.

In the international application WO 9706155 the Synthélabo company discloses derivatives of naphthalen-1-yl-piperazines as ligands of the 5-HT_{1A} receptor useful in treating problems due to withdrawal or to the abuse of stupefacients.

In the application WO 9429293 the firm of Yamanouchi Pharm Co Ltd reports derivatives of fluoro-chromanes as being ligands of the 5-HT_{1A} receptor useful in the treatment of drug dependence.

In the application U.S. Pat. No. 5,741,789 the firm of Eli Lilly and Company describes hetero-oxy-alkanamines as being agonists or partial agonists of the 5-HT_{1A} receptor useful in treating abuse of substances. In the international application WO 0000196, the same firm describes pyrrolidines and pyrrolines as being non-selective antagonists of the 5-HT_{1A} receptor, useful in the treatment of the abuse of substances.

However, in none of the patent applications mentioned above, is the use of 5-HT_{1A} ligands in the treatment of drug dependence on psycho-active substances other than ethanol (WO 9741858) supported by pharmacological data. Furthermore, the 5-HT_{1A} agonists tested clinically in treating dependence on certain stupefacients such as, for example, cocaine, have provided negative results. The impact of compounds with pharmacological activity of the 5-HT_{1A} agonist type on the treatment of drug dependence on opioids is thus, up till now, inexistent. According to the present state of the art, the potential utility of new 5-HT_{1A} agonists in the treatment of opioid drug dependence is difficult to predict.

The inventors have discovered, unexpectedly, that the compounds claimed by the applicant in patent WO 98/22459 and represented by the general formula (I): 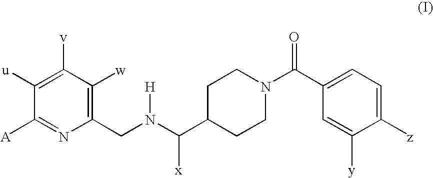
in which:
u represents a hydrogen atom or a methyl radical with the reservation that when u is a methyl radical then v and w represent a hydrogen atom;
v represents a hydrogen atom, a chlorine atom or a methyl radical with the reservation that when v is a methyl radical then u and w represent a hydrogen atom;
w represents a hydrogen atom, a fluorine atom or a methyl radical with the reservation that when w is a methyl radical then u and v represent a hydrogen atom;
x represents a hydrogen atom or a fluorine atom;
y represents a chlorine atom or a methyl radical;
z represents a hydrogen atom or a fluorine atom or a chlorine atom or a methyl radical;
A represents:
   a hydrogen atom or a fluorine atom or a chlorine atom;
   an alkyl radical in C₁-C₅, i.e. a saturated aliphatic hydrocarbon remainder with straight or branched chain, containing from 1 to 5 atoms of carbon such as methyl, ethyl, propyl, butyl, pentyl, isopropyl, 1-methyl-ethyl, 1-methyl-propyl, 1-methyl-butyl, 2-methyl-propyl, 2-methyl-butyl or 3-methyl-butyl, 1-ethyl-propyl, 2-ethyl-propyl;
   a fluoroalkyl radical such as monofluoromethyl (—CH₂F) or difluoromethyl (—CHF₂) or trifluoromethyl (—CF₃) or 1-fluoro-1-ethyl (—CHFCH₃) or 1,1-difluoro-1-ethyl (—CF₂CH₃);
   a cyclopropyl or cyclobutyl or cyclopentyl radical;
   an aromatic heterocyclic group with 5 links, substituted or not, containing 1, 2, 3 or 4 heteroatoms chosen from amongst nitrogen, oxygen and sulphur but nonetheless without more than one oxygen and/or sulphur atom being present in the heterocycle A.
   an alkoxy (R₁O—) or alkylthio (R₁S—) group in which the R₁ radical represents:
   an alkyl radical in C₁-C₅ such as defined above;
   a monofluoromethyl or trifluoromethyl radical;
   a cyclopropyl or cyclobutyl or cyclopentyl radical;
   an amino group of type II 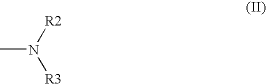
in which R₂ and R₃, identical or different, represent hydrogen or an alkyl radical in C₁-C₅ such as defined above or a cyclopropyl group or a trifluoromethyl group;
   a saturated cyclic amino group of type III 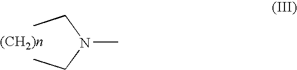
in which n can be the whole numbers 1 or 2;
   an alkoxycarbonyl group, preferably a methoxycarbonyl (CH₃OCO—) group or an ethoxycarbonyl (CH₃CH₂OCO—) group; as well as its addition salts with pharmaceutically acceptable organic or mineral acids are, unexpectedly, potentially efficient in treating opioid drug dependence.

The compounds of formula (I) are known selective agonists of 5-HT_{1A} receptors and their uses in other medical fields are described in the international application WO 98/22459. Preferably, in the present invention, the compound of formula (I) is (3-Chloro-4-fluorophenyl)-(4-fluoro-4-{[(5-methyl-pyridin-2-yl-methyl)-amino]-methyl}-piperidin-1-yl)-methadone fumarate, that is: A=H, u=CH₃, v and w=H, x=F, y=Cl and z=F in the general formula (I). Hereinafter the compound in question is named compound F.

The single figure shows the effect of compound F on morphine dependence.

The validity of animal models for studying the neurobiological and behavioural mechanisms involved in drug dependence date back to over twenty years of research. Several aspects of the behaviour of dependent individuals can thus be reproduced in the laboratory animal. For example, in the rat, made dependent by chronic administration of morphine, the sudden interruption of morphine or the administration of an antagonist of opiate receptors induces a state called hyperalgesia which can easily be documented by means of nociceptives. Thus, this state of hyperalgesia induces and constitutes a sensitive marker, strong and widely recognised as being from opiate dependence. Consequently, the inventors chose to use such a marker to evaluate the capacity of the compounds of the invention to oppose the appearance of a morphine dependence syndrome.

Compound F was compared with gepirone and lesopitron chosen as reference 5-HT_{1A} agonists. Gepirone [83928-76-1], which is a close structural analogue of buspirone [33386-08-2] and of tandospirone [112457-95-1] is a 5-HT_{1A} agonist in phase III clinical trials. Lesopitron [132449-46-8] is the only 5-HT_{1A} agonist in active development in indicating drug dependence (Pharmaprojects March 2001).

The following examples serve to illustrate the pharmacological activity of the compound F and, therefore, its potential utility in the therapeutic aim claimed in the invention.

### EXAMPLE 1

Measurement of the affinity of compound F and reference products for the 5-HT_{1A} receptor

Study of the bond with the 5-HT_{1A} receptor was carried out according to a standard method (Naunyn-Schmiedeberg's Arch. Pharmaco. 1991, 343, 106). The inhibition constants (Ki) of the products according to the invention were estimated from displacement experiments using the version 4 RADLIG non-linear regression programme of EBDA (Equilibrium Binding Data Analysis) (Biosoft, Cambridge, UK, Mc Pherson, 1985). The dissociation constants of the radioactive ligands used in the calculations are 0.31 nmole for ³H-8-OH-DPAT. The values of pKi (−logKi) are provided under the form of average±SEM of at least 3 experiments.

The obtained results are given in the table below.

| | pKi |
|---|---|
| Compound | site 5-HT_{1A}^{a)} |
| compound F | 9.07 |
| gepirone | 7.17 |
| lesopitron | 7.26 |

^{a)}Tissue: cerebral cortex of rat

### EXAMPLE 2

Effects of compound F and reference products on morphine dependence

The products are administered by means of osmotic mini-pumps (model 2ML2; flow rate 5 μl/hr: Alza Corporation, Palo Alto, USA) implanted subcutaneously on the first day of the experiment and extracted two weeks later. The pump is set in place through a transversal incision made in the skin of the dorsal face of the rat, the liberation orifice being directed towards the head.

Morphine chlorhydrate is administered at 5 mg/rat/day (41.7 mg/ml) in solution in distilled water. The compound F is administered at 0.63 mg/rat/day (5.25 mg/ml) in solution in distilled water. Gepirone and lesopitron are administered at 2.5 mg/rat/day (20.8 mg/ml) in solution in distilled water. The pumps implanted in the control animals release 0.12 ml of 0.9% NaCl (saline)/rat/day; the doses referring to the weight of the non salified compound.

The study consists of two phases: a phase of chronic treatment lasting 2 weeks, and a withdrawal phase lasting 1 week.

On the experiment's first day the rats were given implants of two osmotic mini-pumps. Six experimental groups received one of the following treatments: (a) pump 1=saline and pump 2=saline (n=11); (b) pump 1=saline and pump 2=compound F at 0.63 mg/rat/day (n=11); (c) pump 1=morphine at 5 mg/rat/day and pump 2=saline (n=13); (d) pump 1=morphine at 5 mg/rat/day and pump 2=compound F at 0.63 mg/rat/day (n=13); (e) pump 1=morphine at 5 mg/rat/day and pump 2=gepirone at 2.5 mg/rat/day (n=13); (f) pump 1=morphine at 5 mg/rat/day and pump 2=lesopitron at 2.5 mg/rat/day (n=13).

After two weeks of chronic treatment, the withdrawal phase began. After taking base measurements on day 14, all the animals received a sub-cutaneous injection of naloxone at 0.63 mg/kg. Next, 30 minutes, 1 hr, 2 hr, 4 hr, and 8 hr after the injection, another series of measurements was taken. The pumps were extracted after the last measurement at 8 hr after the naloxone injection. A series of measurements was also taken daily 24 hr to 4 days after the naloxone injection. During this withdrawal period, the nociceptive thresholds were measured using the Randall and Selitto method (Arch. Int. Pharmacodyn. 1957, CXI, 409) in which increasing pressure is applied to the back paw until the vocalisation threshold is reached. The results are expressed in grams and a 750 g limit is imposed.

In order to evaluate the nociceptive thresholds, the areas under the curves (AUCs) were determined and processed by a variance analysis (ANOVA). The post-hoc comparisons (i.e. those following the variance analysis when the latter is significant) were made using the Student-Newman-Keuls (SNK) test for comparing groups with each other. A statistically significant effect was defined at p<0.05. The areas under the curves (AUCs) obtained between 30 minutes and 4 days after induction of withdrawal by naloxone are shown in the graph in the appendix.

Results

The results of in vitro bond tests (see table above) show that compound F possesses a strong affinity for the receptor of sub-type 5-HT_{1A}. Furthermore the affinity of compound F for this site is much stronger than those of the reference products: gepirone and lesopitron.

In vivo, the values of the AUCs (see figure), significantly weaker in the animals that received morphine compared to those that received physiological serum demonstrate the expected appearance of morphine withdrawal syndrome [ANOVA, treatment effect, F(5.68)=5.7; p<0.001]. The significant lowering (SNK; p<0.05) of the nociceptive thresholds of the animals that received morphine shows that this syndrome can be objectified clearly by measuring the amplitude of the hyperalgesia which develops during the withdrawal phase. The animals co-treated by both morphine and compound F, contrary to those treated by morphine alone and those treated simultaneously by morphine and gepirone (SNK; p<0.05) or lesopitron (SNK; p<0.05) have AUC values (SNK; p>0.05) statistically comparable with those obtained from the control animals. Compound F does not induce an effect on its own, as the comparable thresholds show (p>0.05) for control animals and those treated with compound F alone. It follows that only those animals co-treated by morphine and compound F did not develop hyperalgesia and thus did not demonstrate any morphine withdrawal syndrome. The 5-HT_{1A} reference agonists (i.e. gepirone and lesopitron) did not have any detectable effect on the hyperalgesia and thus on the morphine withdrawal syndrome. It thus follows that, contrary to the reference compound, compound F is potentially capable of opposing morphine dependence.

This study demonstrates that the compounds of formula (I) together with its addition salts with mineral acids or pharmaceutically acceptable organic acids are, contrary to the 5-HT_{1A} agonists belonging to other chemical classes, potentially useful in treating opioid drug dependence.

A further aim of the invention is pharmaceutical compositions containing as active principle at least one of the derivatives of general formula (I) or one of its salts or hydrates of its salts in combination with one or more pharmaceutically acceptable excipients or vehicles.

The pharmaceutical compositions according to the invention can, for example, be compositions administered by oral, nasal, sublingual, rectal or parenteral means. As an example of compositions able to be administered by oral means, pills, capsules, granules, powders and oral suspensions or solutions can be mentioned.

The appropriate formulations for the chosen method of administration are known and described, for example, in Remington, The Science and Practice of Pharmacy, 19th edition, 1995, Mack Publishing Company.

The efficient dose of a compound according to the invention varies in function of numerous parameters such as, for example, the chosen administration method, the weight, age, sex, the substance or substances responsible for the pathology, and the sensitivity of the individual to be treated. Consequently, the optimum posology must be determined individually, in function of the relevant parameters, by a medical specialist. Even though the efficient doses of a compound according to the invention can vary widely in proportion, the daily doses can be graduated between 0.01 mg and 100 mg per kg of body weight of the person under treatment. Nonetheless, one dose per day of a compound according to the invention, comprised between 0.10 mg and 100 mg per kg of body weight of the person being treated, is preferable.

1. (canceled)

2. (canceled)

3. A method of treating opioid drug dependence comprising administering to a patient in need of such treatment an effective amount of a compound of formula (I) 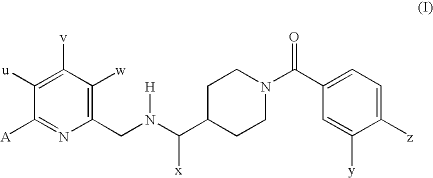
and its therapeutically acceptable salts in which:
u represents a hydrogen atom or a methyl radical with the reservation that when u is a methyl radical then v and w represent a hydrogen atom;
v represents a hydrogen atom, a chlorine atom or a methyl radical with the reservation that when v is a methyl radical then u and w represent a hydrogen atom;
w represents a hydrogen atom, a fluorine atom or a methyl radical with the reservation that when w is a methyl radical then U and v represent a hydrogen atom;
x represents a hydrogen atom or a fluorine atom;
y represents a chlorine atom or a methyl radical;
z represents a hydrogen atom or a fluorine atom or a chlorine atom or a methyl radical;
A represents:
a hydrogen atom or a fluorine atom or a chlorine atom;
an alkyl radical in C₁-C₅, i.e. a saturated aliphatic hydrocarbon remainder with straight or branched chain, containing from 1 to 5 atoms of carbon such as methyl, ethyl, propyl, butyl, pentyl, isopropyl, 1-methyl-ethyl, 1-methyl-propyl, 1-methyl-butyl, 2-methyl-propyl, 2-methyl-butyl or 3-methyl-butyl, 1-ethyl-propyl, 2-ethyl-propyl;
a fluoroalkyl radical such as monofluoromethyl (—CH₂F) or difluoromethyl (—CHF₂) or trifluoromethyl (—CF₃) or 1-fluoro-1-ethyl (—CHFCH₃) or 1,1-difluoro-1-ethyl (—CF₂CH₃);
a cyclopropyl or cyclobutyl or cyclopentyl radical;
an aromatic heterocyclic group with 5 links, substituted or not, containing 1, 2, 3 or 4 heteroatoms chosen from amongst nitrogen, oxygen and sulphur but nonetheless without more than one oxygen and/or sulphur atom being present in the heterocycle A;
an alkoxy (R₁O—) or alkylthio (R₁S—) group in which the R₁ radical represents:
an alkyl radical in C₁-C₅ such as defined above;
a monofluoromethyl or trifluoromethyl radical;
a cyclopropyl or cyclobutyl or cyclopentyl radical;
an amino group of type II 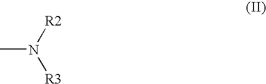
in which R2 and R3, identical or different, represent hydrogen or an alkyl radical in C₁-C₅ such as defined above or a cyclopropyl group or a trifluoromethyl group;
a saturated cyclic amino group of type III 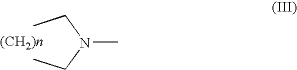
in which n can take the whole numbers 1 or 2;
an alkoxycarbonyl group.

4. The method according to claim 3, wherein A represents a hydrogen atom, u represents a methyl, v and n represent a hydrogen atom, x represents a fluorine, y represents a chlorine and z represents a fluorine.

5. A method according to claim 3, wherein said alkoxycarbonyl group is a methoxy-carbonyl (CH₃OCO—) group or an ethoxycarbonyl (CH₃CH₂OCO) group.
